(19) United States
(12) Patent Application Publication
(10) Pub. No.: US 2004/0170705 A1
(43) Pub. Date: Sep. 2, 2004

(54) Compositions and methods for non-insulin glucose uptake

(76) Inventors: Dusan Miljkovic, San Diego, CA (US); Jovan Hranisavljevic, Belgrade (YU); Zbigniew Pietrzkowski, San Diego, CA (US)

Correspondence Address: Sidley Austin, Brown & Wood, 1501 K Street NW, Washington, DC, 20005, US

(21) Appl. No.: 10/469,384

(22) PCT Filed: Mar. 8, 2002
(86) PCT No.: PCT/US02/07199

(30) Foreign Application Priority Data
Mar. 8, 2001 (US) USO1/07527

(51) Int. Cl. A61K 35/78; C12N 5/08

(52) U.S. Cl. 424/750; 435/366

(57) ABSTRACT

Compositions and methods are provided to increase glucose uptake in a cell. Particularly contemplated compositions include a compound that is isolated from a plant that has AICAR-like activity. In another aspect of the invention, contemplated compositions comprise a non-insulin compound that has AICAR-like activity and activates AMPK in a cell.

This application claims priority to PCT application number PCT/US01/07527 filed on Mar. 8, 2001 incorporated herein by reference in its entirety.

### FIELD OF THE INVENTION

The field of the invention is dietary supplements and related methods.

### BACKGROUND OF THE INVENTION

Elevated blood glucose and blood lipids are a relatively common underlying condition in numerous diseases and may be acquired in various ways. Among other causes, elevated blood glucose levels are frequently precipitated by an altered metabolism associated with a diabetic condition, and treatment of diabetic conditions often includes insulin therapy along with synthetic oral anti-diabetic agents, such as metformin, sulfonylurea, etc. Despite an improvement of some clinical parameters (i.e. reduction of blood glucose to at least some extent) in people with elevated blood lipid and blood glucose, various side effects, including insulin resistance, allergic reactions, etc. may arise from long-term treatment using insulin.

Alternative treatments of diabetes, and especially non-insulin dependent diabetes mellitus (NIDDM), are frequently based on yeast, or derivatives of yeast. Yeast can be grown in the presence of chromium salts, and yeast cells or extracts of cells grown in that manner are particularly rich in “glucose tolerance factor” (GTF), a compound known to enhance the biological effect of insulin. Although some yeast preparations help reduce elevated blood glucose concentrations, in many cases, considerable amounts of yeast preparations must be ingested for a substantial period in order to improve a hyperglycemic condition. Moreover, long-term use of yeast preparations over extended periods tends to become problematic for some patients, especially in patients who have a history of yeast infections. Still further, many crude yeast preparations have a bitter taste that some patients may find objectionable.

To alleviate at least some of the problems associated with yeast preparations, concentrated, de-bittered and freeze dried yeast preparations have been developed. Such preparations are typically in tablet form, and may conveniently be ingested during a meal. However, the relatively high degree of processing of such cells/extracts may reduce the biological potency of the yeast preparation. Moreover, preservatives and additives (e.g., for pressing or otherwise forming of tablets) are typically needed to maintain at least some anti-hyperglycemic activity.

In still other methods of reducing blood glucose on a non-insulin basis, chromium picolinate may be administered. Chromium picolinate is reported to be moderately effective in reducing an elevated blood glucose level in human. However, chromium picolinate exhibits considerable toxicity and may therefore not be generally regarded as safe.

Although various methods of reducing an increased blood concentration of glucose are known in the art, all or almost all of them suffer from one or more disadvantages. Therefore, there is still a need to provide improved compositions and methods to reduce glucose concentration.

### SUMMARY OF THE INVENTION

The present invention is directed to compositions and methods of reducing glucose concentrations in an organism.

In one aspect of the inventive subject matter, a composition is isolated from a plant, has AICAR-like activity, and increases glucose uptake into a cell. The composition is preferred to be insulin independent. The composition is also preferred to have a molecular weight Mw of no more than 2000 and a UV light absorption maximum at about 260 nm. It is further preferred that the composition is present in a medium surrounding the cell at a concentration of between about 2-10 micrograms per milliliter, and that the medium surrounding the cell is depleted of at least one of a nutrient and oxygen. The cell is contemplated to be a myocyte or part of an in vivo tissue such as liver tissue, skeletal muscle tissue, pancreatic tissue, or adipose tissue.

In another aspect of the inventive subject matter, a compound is identical with a molecule isolated from Hordeum vulgare, and has AICAR-like activity, increases glucose uptake into a cell, and the molecule isolated from Hordeum vulgare reduces blood glucose in an organism when the molecule is administered at a concentration effective to reduce the concentration of glucose. It is preferred that the compound is synthesized in vitro and is modified to increase or decrease at least one of the following: an increase in glucose uptake into a cell; solubility in a solvent; chemical stability; and in vivo specificity.

In a third aspect of the inventive subject matter, a compound is a non-insulin compound that has AICAR-like activity and activates AMPK in a cell. It is preferred that the compound is isolated from a Hordeum vulgare plant seed. It is also preferred that the compound increases glucose uptake in a non-insulin dependent manner when the compound is present in a medium surrounding the cell at a concentration of between 2-10 micrograms per milliliter. It is further preferred that the increase in glucose uptake is reduced when the medium includes L-N-mono-methyl-L-arginine at a concentration of 300 micromoles per liter.

In a further aspect of the inventive subject matter, a method of treating a cell is contemplated to include the steps of identifying the cell as having a condition that activates AMPK and presenting the cell with a compound having AICAR-like activity at a concentration effective to modulate at least one of an import, export, or synthesis of a molecule.

Various objects, features, aspects and advantages of the present invention will become more apparent from the following detailed description of preferred embodiments of the invention.

### BRIEF DESCRIPTION OF THE DRAWING

FIG. 1 is a flow diagram showing an exemplary method of reducing blood concentration of glucose according to the inventive subject matter.

FIG. 2 is a flow diagram showing an exemplary method of treating a cell according to the inventive subject matter.

FIG. 3 is a schematic showing an exemplary preparation of contemplated compounds and thaumatin-like proteins.

FIG. 4A is a table depicting reduction of blood glucose concentrations in human volunteers using contemplated compositions according to the inventive subject matter.

FIG. 4B is another table depicting reduction of blood glucose concentrations in human volunteers using contemplated compositions according to the inventive subject matter.

FIG. 5A is a table depicting reduction of blood lipid concentrations in human volunteers using contemplated compositions according to the inventive subject matter.

FIG. 5B is another table depicting reduction of blood lipid concentrations in human volunteers using contemplated compositions according to the inventive subject matter.

FIG. 6 is a graph depicting fermentation rates of yeast incubated with contemplated compounds at anaerobic and aerobic conditions.

FIG. 7 is a table depicting intermediate activities by hours of retentate (high molecular weight) and permeate (low molecular weight) of GMM sample after dialysis.

FIG. 8 is a graph depicting fractions of eluates from the retentate portion of GMM sample.

FIG. 9 is another graph depicting fractions of eluates from the retentate portion of GMM sample.

FIG. 10 is another graph depicting fractions of eluates from the retentate portion of GMM sample.

FIG. 11 is a graph depicting L6 cells treated with insulin in serum-free and serum-supplemented medium.

FIG. 12 is a graph depicting the effect of the permeate portion of GMM sample on glucose uptake in L6 cells.

FIG. 13 is a graph depicting the effect of AICA, L-NMMA, and AICAR+L-NMMA on glucose uptake.

FIG. 14 is a graph depicting the effect of the permeate portion of GMM sample and the effect of the permeate portion+L-NMMA on glucose uptake.

### DETAILED DESCRIPTION

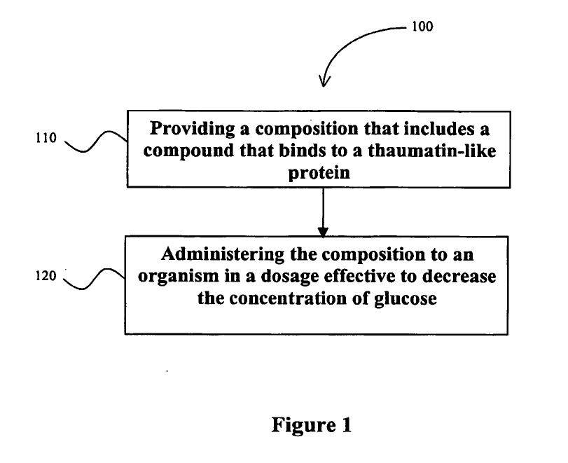

In FIG. 1, a method 100 of reducing a glucose concentration in an organism has a step 110 in which a composition is provided that includes a compound that binds to a thaumatin-like protein. In a subsequent step 120, the composition is administered to the mammal in a dosage effective to decrease the blood concentration of glucose.

As used herein the term “compound that binds to a thaumatin-like protein” refers to any compound or mixture of compounds that exhibit a binding preference to a thaumatin-like protein from barley of at least 10-fold, more preferably at least 100-fold over binding to other barley proteins, wherein binding of contemplated compounds to the thaumatin-like protein will preferably have a K_{D} of less than 10⁻³M, more preferably of less than 10⁴M. The mode of binding need not be limited to a single interaction (e.g., hydrophobic interaction), but may include multiple interactions (e.g., electrostatic interactions and hydrogen bonding, etc.). It is especially contemplated that binding is reversible, however, irreversible binding is not excluded. Although thaumatin-like proteins from barley are generally preferred binding partners for compounds according to the inventive subject matter, thaumatin-like proteins from alternative sources, including microorganisms, plants, and animals are also contemplated. Thaumatin-like proteins are a well characterized class of polypeptides and are described, for example, in Cvetkovic et al., J. Serb Chem. Soc. 62(9):777-786 (1997), Cvetkovic et al., J. Serb. Chem. Soc. 62(1):51-56 (1997) and Cvetkovic et al., J. Inst. Brew. 103:183-186 (1997), all of which are incorporated by reference herein.

As also used herein, the term “elevated glucose concentration” refers to a concentration that is above the clinical range considered normal (i.e., above 110 mg/dl). Similarly, the term “elevated lipid concentration” refers to a concentration of blood lipids that is above the clinical range considered normal.

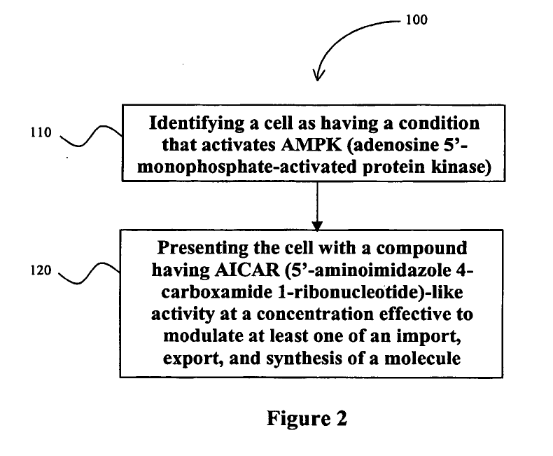

In FIG. 2, a method 100 of treating a cell has a step 110 in which the cell has a condition that activates an AMPK (adenosine 5′-monophosphate-activated protein kinase). In a subsequent step 120, the cell is presented with a compound having AICAR-like activity at a concentration effective to modulate at least one of an import, export, and synthesis of a molecule.

The term AICAR refers to 5′-aminoimidazole 4-carboxamide 1-ribonucleotide. As used herein, the terms “AICAR-like activity” and “5′-aminoimidazole 4-carboxamide 1-ribonucleotide” refer to any activity that stimulates insulin independent glucose uptake into L-6 muscle cells that is inhibited by L-NMMA, and which does not include AICA or AICAR or glucose. Also, contemplated compounds do not include tocol or tocotriene compounds. As used herein, the term “L-NMMA” refers to NG-methyl-L-arginine, acetate salt, which is an inhibitor of nitric oxide synthase (NOS). AMPK has known metabolic regulatory effects on fatty acid and carbohydrate metabolism in liver, adipose tissue, pancreatic beta-cells, and skeletal muscle. AMPK is known to effect increases in glucose uptake and increases in fatty acid oxidation in skeletal muscle, increases in fatty acid oxidation, decreases in cholesterol synthesis, and decreases in lipogenesis in the liver. Furthermore, AMPK is known to modulate insulin secretion in pancreatic islets.

It is contemplated that the composition is a pharmaceutical or nutraceutical for treatment of diabetes. In a preferred aspect of the inventive subject matter, the composition is prepared from Hordeum vulgare (as outlined in examples, infra), and orally administered in 3 daily doses of 500 mg, respectively, to a human diagnosed with non-insulin dependent diabetes mellitus (NIDDM). Thus, especially preferred compositions include a compound that binds to thaumatin-like proteins and that reduces a concentration of glucose in an organism when the compound is administered to the organism at a concentration effective to reduce the concentration of glucose.

In alternative aspects of the inventive subject matter, it is contemplated that appropriate compositions and compounds need not be limited to a preparation from Hordeum vulgare, but may also include preparations from various plants other than Hordeum vulgare, and particularly contemplated alternative plants include Hordeum spec., and members of the poaceae family. While the preparation of contemplated compositions and/or compounds is preferably from plant extracts, it should further be appreciated that contemplated compositions and/or compounds may also be isolated from microorganisms (i.e., bacteria, fungi, yeasts, unicellular eucaryotic organisms) or animals, so long as contemplated compounds bind to a thaumatin-like protein and reduce a glucose concentration in an organism.

In contemplated embodiments of the inventive subject matter, contemplated compositions are identical with, or a derivative of, a molecule isolated from Hordeum vulgare. Derivatives can be advantageously developed to address issues of bioavailability, targeting and other specificity, efficacy, and reduction in toxicity. It is further contemplated that the molecule may be isolated from Hordeum vulgare through any procedure including malting, mashing, salt extraction, a buffer extraction, ethanol extraction, anion exchange chromatography, and molecular sieving. Still further, it is contemplated that the molecule is isolated from any part of the Hordeum vulgare or a plant seed of Hordeum vulgare. It is preferred that the molecule is isolated from a malted seed of Hordeum vulgare.

In still further alternative aspects, it should be appreciated that contemplated compounds may be isolated, purified to homogeneity, and the structure be elucidated. Consequently, it should be appreciated that contemplated compounds and/or compositions may be entirely (de novo) or partially synthesized/modified in vitro. For example, where contemplated compounds are partially synthesized, a precursor of contemplated compounds may be isolated from a plant or microorganism, and then be subjected to one or more steps to arrive at contemplated compounds. Alternatively, contemplated compounds may be modified in one or more synthetic steps to impart a particularly desirable physico-chemical property. For example, contemplated compounds may be esterified with a polar compound (e.g, polyethylene glycol) to increase water solubility. In another example, contemplated compounds may be coupled to a resin or other material to control the rate of release to the organism.

Preferred contemplated compounds have a relatively low molecular weight, typically no more than 2000 Da, however, it should be recognized that the molecular weight may vary considerably and will predominantly depend on the source from which the compound is isolated, synthetic modifications, dimerizations and multimerizations. Likewise, it is contemplated that suitable compounds need not be limited to compounds having a UV absorption maximum at about 260 nm (which is characteristic for contemplated compounds isolated using the procedure outlined below), and various spectral characteristics other than a UV₂₆₀ peak are also suitable. Similarly, while contemplated compounds isolated from Hordeum vulgare are soluble in a lipophilic solvent at a concentration of at least 10 mg per milliliter, higher or lower solubilities are also contemplated and will typically depend on the source from which contemplated compounds are isolated, and/or on further chemical modifications of contemplated compounds. The term “lipophilic solvent” as used herein includes all solvents that have a miscibility with H₂O of less than 10 vol %.

While it is generally preferred that contemplated compounds are chemically substantially pure (i.e., concentration of contemplated compounds greater than 90 wt %, preferably greater than 95 wt %, most preferably greater than 99 wt %/o), it should also be appreciated that contemplated compounds may be coupled to one or more than one molecule, and particularly contemplated molecules include thaumatin-like proteins. Thus, contemplated compositions include complexes between contemplated compounds and thaumatin-like proteins, and especially include complexes between contemplated compounds and thaumatin-like proteins as they are isolated from the appropriate sources (infra).

With respect to the glucose concentration, it is generally contemplated that the glucose concentration is a blood glucose concentration. However, further contemplated glucose concentrations also include concentrations of glucose covalently or non-covalently bound to molecules found within the organism, and especially contemplated alternative glucose concentrations include concentrations of glycosylated proteins (e.g., glycosylated hemoglobin or collagen).

While it is generally contemplated that suitable thaumatin-like proteins are isolated from Hordeum vulgare, alternative thaumatin-like proteins are also contemplated and include thaumatin-like proteins isolated from microorganisms, plants, and animals, which may or may not be expressed in a recombinant system. There are various protocols for isolation of thaumatin-like proteins known in the art (see e.g., Barre et al, Purification and structural analysis of an abundant thaumatin-like protein from ripe banana fruit. Planta. 2000 Nov;211(6):791-9; Oh, et al., Isolation of a cDNA encoding a 31-kDa, pathogenesis-related 5/thaumatin-like (PR5/TL) protein abundantly expressed in apple fruit. Biosci Biotechnol Biochem. 2000 February;64(2):355-62; Tattersall, et al. Identification and characterization of a fruit-specific, thaumatin-like protein that accumulates at very high levels in conjunction with the onset of sugar accumulation and berry softening in grapes. Plant Physiol. 1997 July; 114(3):759-69), and all the known protocols are considered suitable for use in conjunction with the teachings presented herein.

It should be especially appreciated that contemplated compositions not only reduce elevated blood glucose concentration in human suffering from NIDDM, but may also reduce blood glucose concentrations in individuals having elevated blood glucose concentrations for reasons other than NIDDM, including obesity, dietary effects, etc. It is especially contemplated that individuals with or without NIDDM will have a blood glucose concentration of at least 90 mg/dl, more preferably of at least 120 mg/dl, and most preferably of at least 200 mg/dl.

Furthermore, contemplated compositions have also been shown to advantageously reduce elevated blood lipid concentrations (infra), wherein blood lipids particularly include triglycerides, fatty acids, HDL-cholesterol, and LDL-cholesterol, and it is further contemplated that the reduction of blood lipids may be concomitantly with the reduction of blood glucose levels, or independent of the reduction of the blood glucose level.

In further aspects of the inventive subject matter, it should be appreciated that contemplated compositions may further comprise active or inactive ingredients, including compositions known to decrease a blood lipid concentration, and/or compositions known to decrease blood sugar concentrations. For example, alternative compositions may include at least one of a tocol, vitamins, and/or mineral preparations, GTF, metformin, sulfonylurea, and the like. Inactive ingredients include fillers, coloring agents, stabilizers, and the like.

Thus, an exemplary method of treating a person (e.g., diagnosed with NIDDM) having an increased blood concentration of glucose of approximately 150 mg/dl, and an increased blood concentration of total cholesterol of above 280 mg/dl, or more has one step in which contemplated compositions are provided. In a further step, the composition is administered to the person in a dosage effective to decrease the concentration of glucose.

With respect to the blood glucose level it is contemplated that a treatment according to the inventive subject matter need not be limited to blood glucose levels of approximately 150 mg/dl, but may also be indicated at many blood concentrations of glucose above 70-110 mg/dl. Although not wishing to be bound to a particular theory or mechanism, it is contemplated that the reduction in the blood glucose level may be due to an enhanced glucose uptake into the cell. However, it should be noted that compositions according to the inventive subject matter are non-GTF compositions. The duration for contemplated treatments may vary significantly, and suitable durations may be within the range of a single dose, but also for a predetermined period, including one week, several weeks, several months, and even several years. Consequently, it should be appreciated that compositions according to the inventive subject matter may also be prophylactically administered to a human to prevent hyperglycemia, or some form of dyslipidemia.

In still further aspects of the invention, contemplated compositions increase glucose uptake into a cell. It is contemplated that the cell may be any cell including a myocyte, or part of a tissue. It is further contemplated that the cell may be insulin dependent or insulin independent. Contemplated tissues are preferred to be in vivo, and may include liver tissue, skeletal muscle tissue, pancreatic tissue, and adipose tissue. It is further preferred that the cell is a myocyte and is insulin independent.

Contemplated compositions may be present in a medium surrounding the cell, such as the serum or interstitial fluids. It is further contemplated that the composition may increase glucose uptake into a cell when the compound is present in a medium surrounding the cell at a concentration of between 2-10 micrograms per milliliter. Furthermore, it is contemplated that the cell or the medium surrounding the cell is depleted of at least one of a nutrient and oxygen. The term “depleted” refers to less than 10% of a normally fit cell or less than 15% of a particular nutrient (i.e. glucose). Moreover, it should be appreciated that an increase in glucose uptake is reduced when the medium surrounding the cell comprises L-N-mono-methyl-L-arginine at a concentration of 300 micromoles per liter.

It is further contemplated that the composition may be modified to increase or decrease an increase of glucose uptake into a cell. Furthermore, it is contemplated that the composition may be modified to increase or decrease solubility in a solvent, chemical stability, or in vivo specificity.

In further alternative aspects of the inventive subject matter, the composition may also be administered to an organism other than a human, and particularly preferred alternative organisms include livestock (e.g., cattle, pigs, horses, etc.) and pets (e.g, dogs, cats, rodents, birds, etc.). With respect to contemplated compositions, the same considerations as described above apply.

It is especially contemplated that treatment according to the inventive subject matter may also result in significant weight loss, particularly in persons with obesity, NIDDM, or other condition associated with increased body weight. It is generally contemplated that the treatment according to the inventive subject matter is not limited to reduction of blood glucose alone, but may concomitantly (or by itself) include reduction of a particular lipid or lipid group. For example, slightly elevated total cholesterol (e.g., 220 mg/dl) may be an indication for treatment with the contemplated compounds. Alternatively, it is contemplated that an imbalance between HDL and LDL (i.e. LDL>>HDL) may be normalized employing a treatment according to the inventive subject matter. Similarly, while the total cholesterol in the patient need not be elevated, treatment with the contemplated method may still be indicated due to an elevated triglyceride level.

With respect to the dosage, form, and route of administration it is contemplated that there are many alternative oral preparations besides 3 oral daily doses of 500 mg. For example, where relatively high dosages are required, dosages may increase from 500 mg-5 g per day, and more. High dosages may also be required where the potency of an extract is relatively low. Likewise, in cases where low dosages (e.g., maintenance therapy) are required, or the extract has a comparably high potency, daily dosages between 500 mg and 25 mg, or less, are appropriate. Therefore, it is generally contemplated that among other parameters the patient's particular condition and the potency of the preparation will at least partially determine the frequency of application. For example, where high dosages are to be administered to the patient, more than 3 daily dosages are contemplated, including 4-6 and more. Where low dosages, especially dosages lower than 500 mg/day are contemplated, single, bidaily, or less frequent administrations are appropriate.

Of course it should also be recognized that the form of administration may vary considerably. For example, oral administration need not be limited to a tablet, and alternative oral administrations may include powders, gel-caps, syrups, gels, etc. Where oral administration is not desirable, it is further contemplated that alternative routes are also appropriate, including injections, transdermal, pulmonary or intranasal delivery.

### EXAMPLES

The following examples provide various experimental procedures to make and use contemplated compounds according to the inventive subject matter. Examples 1 and 2 describe basic and improved procedures of producing compositions according to the inventive subject matter, respectively. The biological activity of the compounds isolated according to procedures in Examples 1 and 2 is described in Example 3 and 4, and Example 5 provides experimental support for specific binding of contemplated compounds to thaumatin-like proteins. Example 6 provides experimental procedures to determine the molecular weight of molecules in GMM. Example 7 provides experimental data for chromatographic fractions of GMM retentate, and example 8 provides experimental support for an increase of glucose uptake in L6 muscle cells of GMM permeate.

### Example 1

Barley grains were malted according to procedures well known in the art of beer brewing (see e.g., Principles of Brewing Science, Second Edition, by George J. Fix; Brewers Publications; ISBN: 0937381748, or The Brewers' Handbook by Ted Goldhammer; KVP Publishers; ISBN: 0967521203). In order to extract soluble substances from the malt and to convert additional insoluble solids into soluble material through controlled enzymatic conversion, a step of mashing was subsequently applied to the ground malt (suspended in water) according to a typical brewer's schedule. The temperature cycles were as follows: Incubation at 40° C. for 60 min, incubation at 50° C. for 60 min, incubation at 60° C. for 60 min, incubation at 72° C. for 60 min, and incubation at 75′-80° C. for 60 min. Soluble portions of samples were separated from husks and other insoluble material and freeze-dried.

The freeze-dried barley extract obtained after mashing at 40° C. served as base for fractionation into its components. A first fractionation was achieved by preparative liquid chromatography using a DEAE-Sephacel column (2.6×20 cm) equilibrated with 50 mM phosphate buffer, pH 7.8. 150 mg of the freeze-dried sample was dissolved in 10 ml of buffer and placed on the column. A linear NaCl-gradient (0-0.5 M) was run at a flow rate of 10 ml/h. Fractions (2 ml each) were collected, and elution was monitored at 280 nm. The DEAE chromatography resulted in four distinct protein peak fractions: I—basic, II—neutral, III— and IV—acidic. Respective peak fractions were collected, desalted and concentrated by membrane ultra-filtration using a membrane cut-off pore size of 1000 Dalton, and concentrated corresponding fractions were checked for their capacity to influence yeast fermentation rate. The basic fraction I produced significant inhibitory effect (i.e., a reduction of the yeast fermentation rate), while the remaining three concentrated fractions were almost inert. As it could later be identified (data not shown), the main proteinaceous component in fraction I represent thaumatin-like proteins. It has been noticed during the membrane ultra-filtration of the pooled protein fractions I-IV (i.e., fractions obtained by ion exchange chromatography), that the filtrate of some fractions contains LMW (low molecular weight) substances with a UV absorbance maximum of approximately 260 nm. These observations prompted us to employ molecular sieving chromatography to separate these LMW substances from proteins in these fractions.

For that purpose, the four separated fractions by DEAE-Sephacel column I-IV were pooled and freeze-dried. Molecular sieving chromatography was performed on Sephadex G-75-50 column (2.8×80 cm) with 50 mM phosphate buffer, pH 7.8, containing 0.5 M NaCl (flow rate—12 ml/h, fractions 2 ml, elution recorded at 260 nm). LMW compounds with an absorbance near 260 eluted at relatively high elution volume. Where the separated fractions were individually subjected to molecular sieving on a Sephadex G-75-50 column, LMW compounds eluted near to the end of the separation, typically between 60th-80th fractions. These fractions were designated GMM-1, GMM-2 and GMM-4, and consist of LMW components.

All of GMM-1, GMM-2 and GMM-4 enhanced yeast fermentation, bound strongly and reversibly to thaumatin-like protein (bind to thaumatin-like proteins at low salt condition and release from thaumatin-like proteins at high salt condition), and reduced elevated blood glucose concentration and elevated blood lipid concentration in human diagnosed with NIDDM.

### Example 2

20 g of malted barley flour was suspended in 80 ml of water and stirred over night at ambient temperature. The suspension was supplemented with 120 ml of 0.8 M NaCl solution and salt extraction was continued for 24 hours with stirring. An aqueous extract was separated from the suspension by vacuum filtration over a cellulose filter pad. Alternatively, citrate or other buffers are also contemplated suitable for preparation of an aqueous extract.

The filtered extract was freeze-dried or vacuum-evaporated. So obtained dry malt extract (yield approx. 12-14 g) contained 5.6 g of NaCl originating from the extracting solvent and a complex mixture of water-soluble barley components. The filtered freeze-dried extract was purified by extraction with two 50 ml portions of warm ethanol under vigorous mixing for two hours. The ethanolic extracts were filtered, combined, and evaporated to an oily residue in vacuum. The oily residue was re-dissolved in 15 ml of water and freeze-dried, resulting in a hard glassy yellowish product in a total amount of approx. 3

The glassy yellowish product enhanced yeast fermentation, bound strongly and reversibly to thaumatin-like protein (bind to thaumatin-like proteins at low salt condition and release from thaumatin-like proteins at high salt condition), and reduced elevated blood glucose concentration and elevated blood lipid concentration in human diagnosed with NIDDM.

Thus, it should be recognized that contemplated compositions comprise a plant seed extract (preferably from Hordeum vulgare), wherein the plant seed is malted (preferably at a temperature between about 30° C. and 65° C.) and the extract is prepared from the malted plant seed using a protocol that includes an aqueous extraction step (e.g., using an aqueous buffer such as a citrate buffer), and that the extract reduces a glucose concentration in an organism when the extract is administered to the organism at a concentration effective to reduce the concentration of glucose.

### Example 3

The biological activity of LMW fractions from Example 1 (GMM-1, GMM-2 and GMM-4) and the glassy yellowish product from Example 2 was monitored by quantification of brewers' yeast fermentation rate under anaerobic conditions using a modified Warburg method (Mirsky, N. et al., J. Inorg. Biochem. 13(1):11-21 (1980), which is incorporated by reference herein.

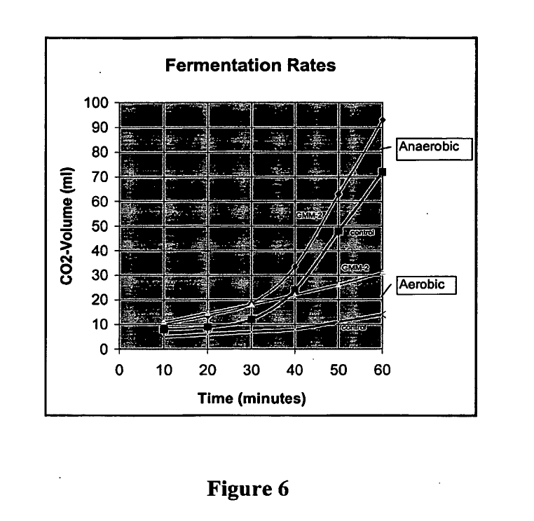

Two grams of wet brewers yeast cells (about 20% dry weight) were suspended in fermentation medium (25 ml of 60 mM phosphate buffer, pH 5.7 and 10 ml of 5% (w/v) glucose solution), and aliquots of the products from example 1 or 2 were added to the fermentation medium for testing. Incubations were carried out in 50 ml fermentation flasks at 25° C. for 60 minutes. The fermentation rates were measured from the volume of generated CO₂. All of the tested LMW fractions or the product from Example 2 showed significant biological activity or bioactivity in that they increased the yeast fermentation rate in the range of about 20-40%. As used herein, a bioactive compound is one that increases or decreases fermentation. In a further experiment, the activity of GMM-2 was checked at aerobic conditions. Despite general restriction of yeast fermentation caused by combined effects of NaCl from buffer and air oxygen (Pasteur effect), the relative amount of generated CO₂ was doubled in comparison to the included control. The comparative results for GMM-2 fraction at anaerobic and aerobic conditions are shown below in FIG. 6. The results conclusively prove modulating activity of the isolated LMW substances on yeast metabolism.

### Example 4

The product obtained in Example 2 was examined for use in humans diagnosed with NIDDM. 25 men were recruited from an outpatient clinic (Endocrinology Department). Mean age within the group was 51 yr, ranging from 36 to 74. Medical records were screened to exclude diabetics taking insulin or oral hypoglycemic agents. All of the subjects agreed to maintain their usual eating habits and health-related behaviors throughout the study. The experimental treatments were run over a period of six month. The participants were instructed to take the preparation in 3 oral daily doses of 1,000 mg each in a tablet form.

All subjects were tested for plasma glucose, glucosylated hemoglobin HbAc1, triglycerides and cholesterol before supplementation and throughout the study at biweekly or monthly intervals depending on type of tests. The subjects were subdivided into groups according to patterns given below:

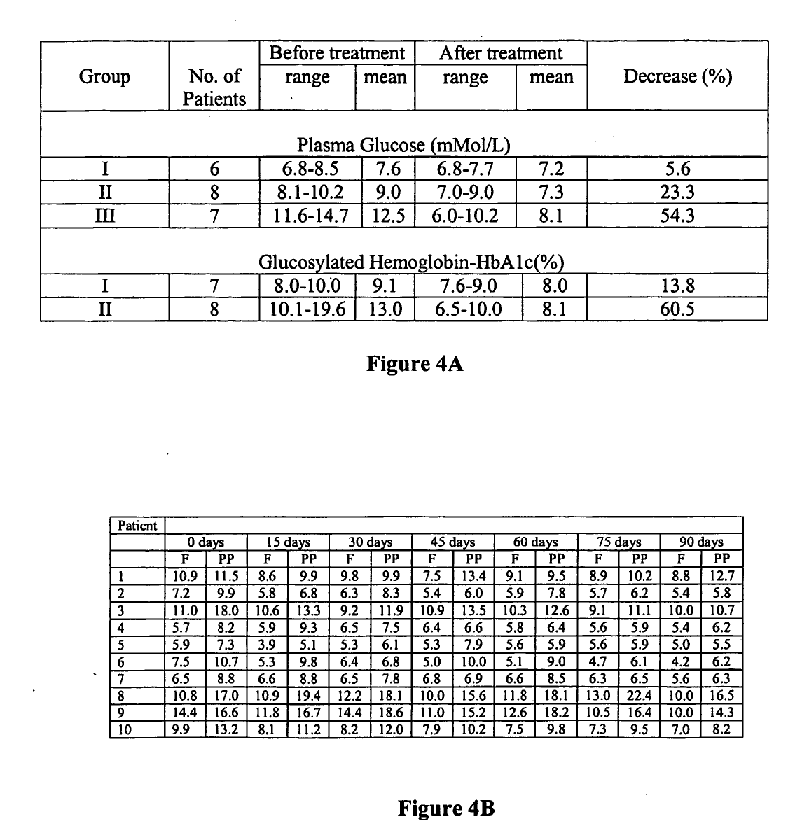

Plasma glucose: According to the plasma glucose levels the subjects were subdivided in three groups for differentiation of the effects: I—up to 8 mMol/L; II—8-10.5 mMol/L and III—above 10.5 mMol/L of plasma glucose concentration. Glycosylated hemoglobin (HbAc1): According to the HbAc1 levels the subjects were divided in two groups: I—below 10% and II—above 10% of the modified hemoglobin. The test results related to glycemia, before and after treatment, are shown in FIG. 4A.

A further set of clinical studies was performed with 10 human volunteers following a similar protocol as outlined above. In this second experiment, blood glucose was measured fasting and postprandial over a period of 90 days, and the results are shown in FIG. 4B. As can be clearly seen, administration of contemplated compounds results in a decrease of fasting and/or postprandial blood glucose of at least 5%, more typically of at least 10%, and most typically of at least 20%. Similarly, the levels of glycosylated hemoglobin was reduced after administration of contemplated compounds at least 5%, more typically at least 20%, and most typically at least 50%.

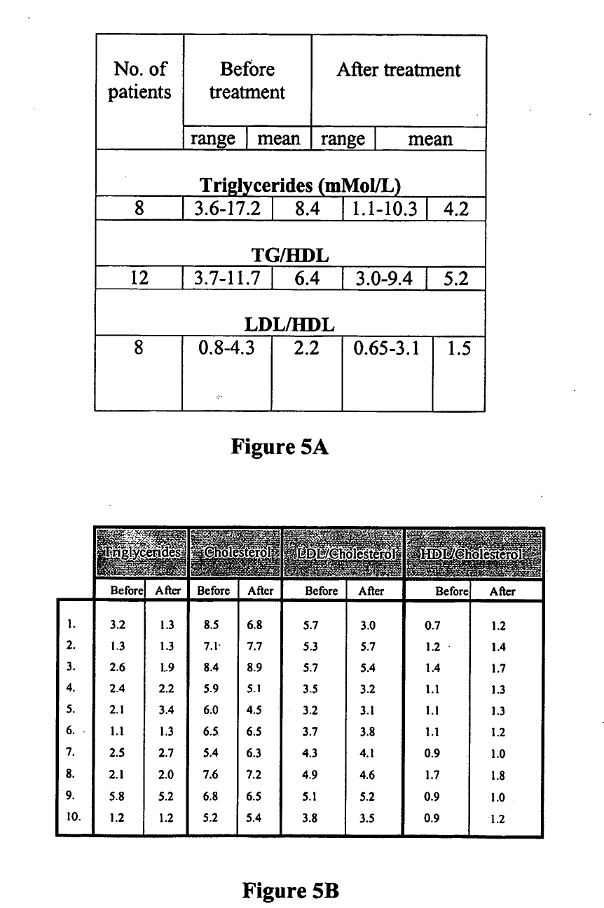

The lipid status of the subjects diagnosed with NIDDM was determined before and after treatment by testing plasma level of triglycerides, and cholesterol (as total, LDL and HDL form). The test results shown in FIGS. 5A and 5B include subjects with disturbed lipid metabolism due to diabetic disease.

The lipid status of the subjects as shown in FIG. 5A includes plasma levels of triglycerides, the ratio of triglycerides over total cholesterol, and the ratio of LDUHDL. The latter two ratios are known as atherosclerotic risk factors. As can be seen from FIG. 5A, administration of contemplated compounds resulted in a reduction of triglycerides of up to 50%, and a significant reduction of about 1-20% of the ratio of triglycerides to HDL cholesterol, with an even more dramatic reduction of the ratio between LDL to HDL cholesterol (about 40%). The lipid status as shown in FIG. 5B includes further results of ten test patients after administration of contemplated compounds and/or compositions over a period of 90 days.

### Example 5

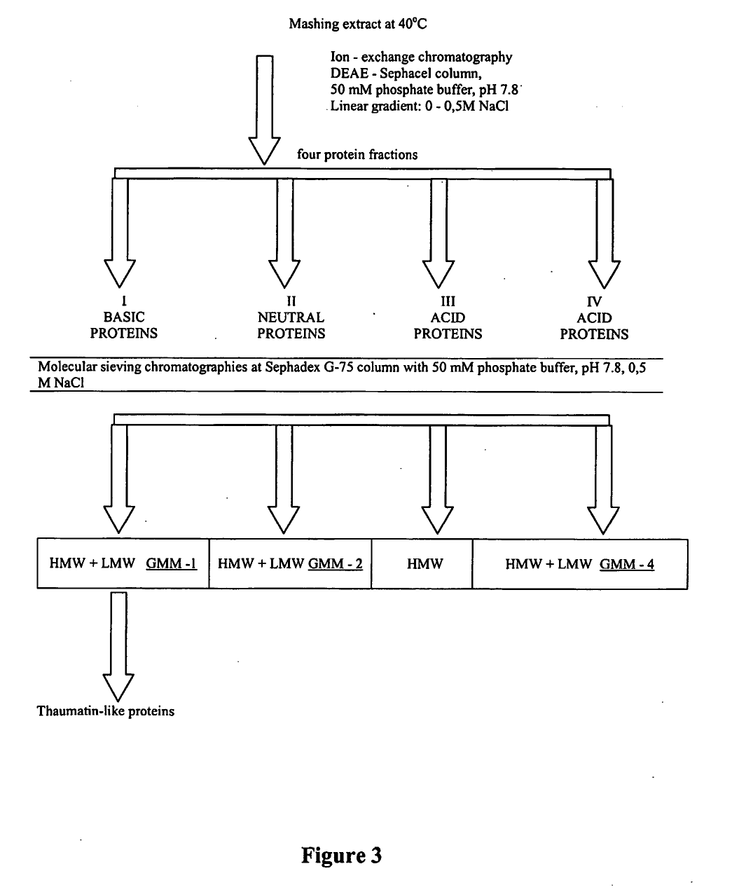

Thaumatin-like proteins were prepared following the procedure as generally outlined in Example 1 and FIG. 3. So isolated thaumatin-like proteins were subjected to repeated molecular sieving in a membrane concentrator using a membrane with a molecular weight cut off of about 1000 Dalton. After a first round of filtration of the protein preparation, 99 ml of buffer (50 mM phosphate buffer, pH 7.8, 0.5 M NaCl) were added to about 1 ml of retentate (i.e. the thaumatin-like protein fraction), and three subsequent rounds of filtration were performed with the same buffer to remove remaining GMM-compounds (i.e., herein presented compounds that reduce elevated glucose) from the thaumatin-like protein preparation. UV absorbance of the filtrate was monitored at 260 nm and the biological activity of sample volumes from the filtrate was tested according to protocols outlined in Example 3. Such prepared thaumatin-like proteins were desalted by membrane filtration employing NaCl-free buffer (50 mM phosphate buffer, pH 7.8), and further used in the following procedure:

To 1 ml of a desalted thaumatin-like protein solution (10 mg/ml), 1.0 ml of a GMM-1 solution (1 mg/ml) was added, and the mixture was incubated at room temperature for 2 hrs. After 2 hrs, 98 ml of 50 mM phosphate buffer, pH 7.8 were added to the mixture and unbound GMM-1 was removed by 3 subsequent rounds of ultrafiltration (each round 1:100 by volume) with buffer.

The thaumatin-like protein with the bound GMM-1 was labeled Sample 1. Sample 1 was then subjected to a molecular sieving chromatography using a Sephadex G-75 column with 50 mM phosphate buffer, pH 7.8, 0.5 M NaCl as solvent, in which a low molecular weight fraction eluted with an absorbance of 260 nm separate from a higher molecular weight fraction of the thaumatin-like protein with absorbance of 280 nm. The low molecular weight fraction was concentrated, desalted, and brought to a volume of 1.0 ml and labeled Sample 2. Samples 1 and 2 were then tested for biological activity employing a procedure as outlined in Example 3. While Sample 1 did not increase the rate of fermentation, Sample 2 significantly increased the rate of fermentation in both aerobic and anaerobic experimental conditions, thereby clearly demonstrating the reversible binding of GMM-1 to a thaumatin-like protein. The same procedure was repeated with GMM-2 and GMM-4. The obtained results were similar to the presented GMM-1 experiment.

Thus, specific embodiments and applications of compositions and methods to reduce glucose concentrations in an organism have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises”, and “comprising”, should be interpreted as referring to elements, components, or steps in a non-exclusive manner, indicating that the referenced elements, components, or steps may be present, or utilized, or combined with other elements, components, or steps that are not expressly referenced.

### Example 6

GMM was dialyzed in order to determine the molecular weight distribution of active GMM components and to purify and concentrate the active components. A series of experiments was performed in portions of 3 g of GMM preparation per one tube. GMM samples were dissolved in 40 ml of water and dialyzed against 500 ml of water for 24 hours. External water was replaced with fresh water after 24 hours, for better separation. The external water portions were combined, concentrated in a vacuum, centrifuged, and evaporated to dryness, resulting in permeate. The contents of the dialysis tubes were centrifuged and evaporated to dryness in a vacuum, resulting in retentate.

The percentage ratio of permeate to retentate was 58.5:25.1, with 16.4 corresponding to insoluble materials from both retentate and permeate. This insoluble part was discarded.

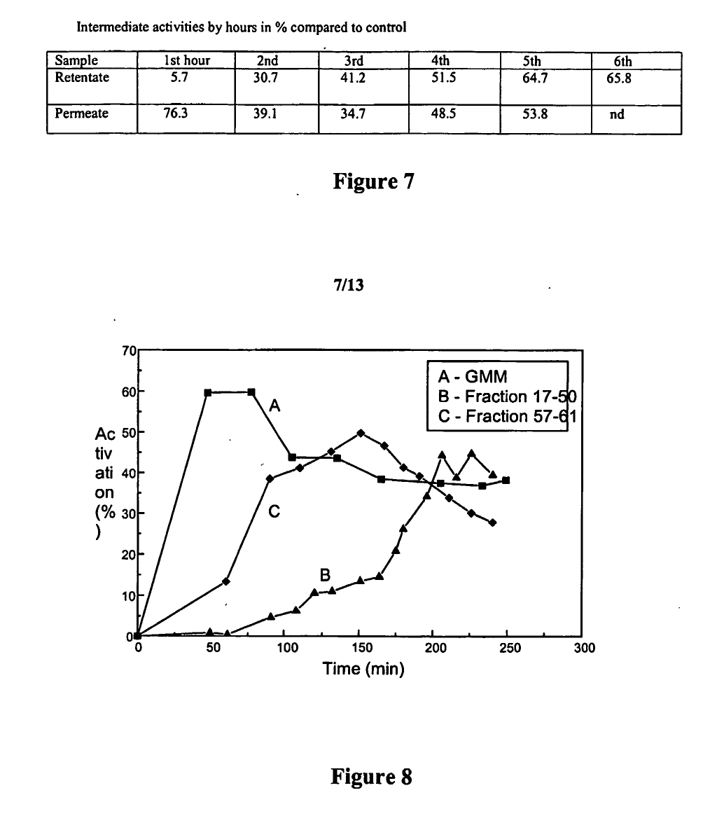

The kinetics (rate of action) of the two soluble fractions in yeast fermentation is shown in FIG. 7. FIG. 7 shows that at least two types of active compounds in GMM are present. These two types include: high molecular weight (HMW, retentate) substance(s) with an increase of activity with the progress of the fermentation process, and low molecular weight (LMW, permeate) substance(s) with an early activity decreasing with progress of fermentation. In addition, the permeate kinetic is overlapped with retentate activity. This may have been due to the nononselective pore size of the dialysis tubing may have allowed for partial passage of retentate components through the pores. This phenomenon has been repeated in repetitive dialysis cycles. The results indicate that the molecular weight of the permeate compounds is close to 2,000 Daltons.

Moreover, the retentate activity results in a significant increase of yeast biomass in a range of 44% over the control. The results may indicate that the retentate GMM fraction acts at a gene level leading to cell multiplication, while the permeate GMM fraction most probably exerts its action through an enhanced glucose uptake in a non-insulin dependent pathway.

### Example 7

The retentate sample from Example 6 was subject to gel permeation chromatography with Sephadex G-75-50 at column of 2×80 cm using 50 mM phosphate buffer, pH 7.8. The GMM/R samples were 40-50 mg dissolved in the buffer. The eluates were fractionated in the fraction collector in 15 minutes intervals, giving 1.5 ml fractions. The elution was traced by spectrophotometric measurement at and absorbed at 280 nm.

Yeast activity tests were then run using Barometric methods, which were more convenient than Gravimetric methods. The tests were run parallel and simultaneous to the control. The eluted fractions were then pooled in three successive sections: Section 1—fractions 17-50; section 2—fractions 51-60; and section 3—fractions 61-70.

An aliquot of fraction 17-50 was used to test for yeast activity, shown in FIG. 8. The active components in FIG. 8 were characterized with late activity development. The early position of the elution pattern in FIG. 8 shows indicates that the size of the components in fraction 17-50 was above 12,000 d. The remaining aliquots of fraction 17-50 was dialyzed against water in dialysis tubing with a 12,000 d cut-off. The retained content in the tube was analyzed for yeast activity and showed relatively strong inhibition, indicating the presence of active components with a molecular weight ranging between 2000 and 12,000 d.

An aliquot of fraction 51-60 was also tested for yeast activity and resulted in early activity development. Additionally, an aliquot of fraction 61-70 was tested for yeast activity and resulted in the strongest inhibitory effect among the three fractions. These data are not shown in the figures.

The eluted fractions were then pooled in five successive sections:

Section 1—fractions 18-32

Section 2—fractions 33-46

Section 3—fractions 47-56

Section 4—fractions 57-61

Section 5—fractions 61-80

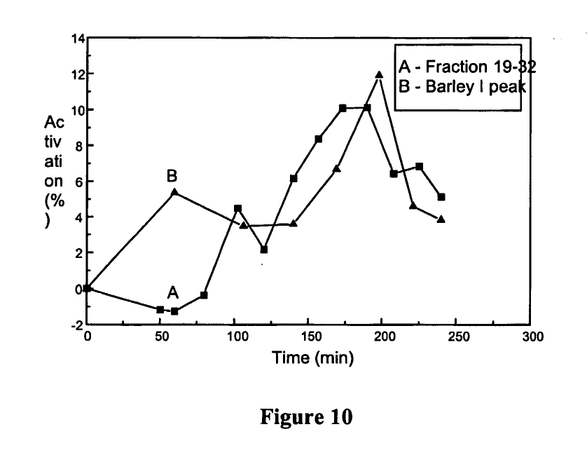

The sections were further analyzed for yeast activity. Fractions 19-32 showed a late activity development, as seen in FIG. 10. That result indicates that fraction 19-32 may have a high molecular weight above 20,000 d. A matching protein in the brewer's barley extract elutes at the same position on GP chromatography. That protein is not abundant in the extract, and has a molecular weight above 12,000 d because it does not release its activity on dialysis with 12,000 d cut-off tubings. The yeast activity profiles of two leading proteins are shown comparatively in FIG. 10.

Fractions 33-46 and fractions 47-56 were tested for yeast activity and showed an inhibitory effect.

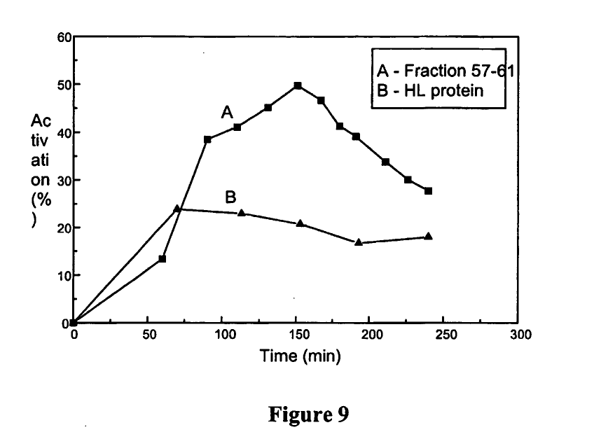

Fractions 57-61 were tested for yeast activity and showed early activity development, as seen in FIGS. 8 and 9. The position of this protein coincides with the position of brewer's barley HL-protein known to carry UV absorbing ligands at 265 nm. Past tests have shown that the HL-protein releases its ligands in the presence of salt in a 0.5M concentration. The HL-protein has a molecular weight of 14,000 d. FIG. 9 shows comparative yeast fermentation activities of HL-protein and its matching component of GMM at Fractions 57-61. The amount of HL-protein in the test was as low as 5 mg.

Thus, chromatographic fractionation of GMM retentate by gel permeation size exclusion revealed the presence of macromolecular species in the GMM preparation, most of which were proteins. Some of the proteins in the GMM retentate showed a significant inhibiting action on the yeast fermentation. Two macromolecular components were detected with apparent yeast fermentation activity. A peak in activity was located in the first portion of an elution pattern, which belonged to a molecular species passing through dialyzing membranes with a 12,000 d cut-off.

### Example 8

The permeate portion of GMM was tested for glucose uptake in L6 muscle cells. L6 cells were grown as monolayers in Dulbecco's modified Eagle's medium (DMEM) containing 1 mM glutamine, 1 mM pyruvate and 25 mM glucose. The medium was supplemented with 5% fetal calf serum (FCS). Experiments were undertaken in 48-well plates seeded from pre-confluent flasks with 50 000 cells per well. The cells were grown to confluence and the medium was changed to DMEM containing 0.5% FCS and 2.5 mM glucose (L6 Myo Medium) for at least 76 hrs to induce differentiation of L6 cells and formation of L6 myotubes. Myotubes were then incubated with test substances for 30 minutes—Protocol SMM, or in Dulbecco's PBS supplemented with 20 mM Hepes and 2 mM glucose without serum—Serum-free Medium (SFM) Protocol as described by Fryer et al. ((Activation of glucose transport by AMP-activated protein kinase via stimulation of Nitric oxide synthase, Diabetes, 49(12), 1978-85, 2000, Fryer LGD, et al).

Next, 30 uM of 2-NBDG was added for 2 minutes at 22C. Subsequently, cell culture medium was removed and cells were washed twice with cold DPBS. Then, cells were lysed in 0.5% SDS. Fluorescence was measured at 466/542 nm (excitation/emission) using 100 ul of cell lysate in 2 ml of water.

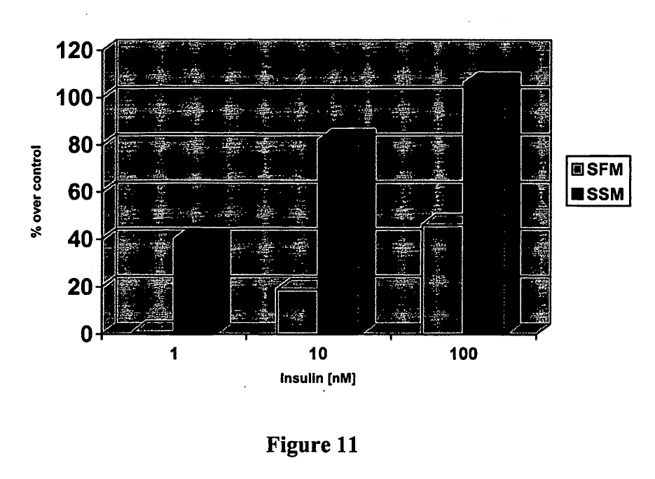

In FIG. 11, L6 cells treated with insulin in serum-free medium (DPBS) responded up to 40-50% glucose uptake increase. However, treatment of L6 with insulin in culture medium supplemented with 0.5% FCS(SSM) increased glucose uptake up to 100-120% by average. These results may suggest that muscle cells are more sensitive to insulin in presence of serum.

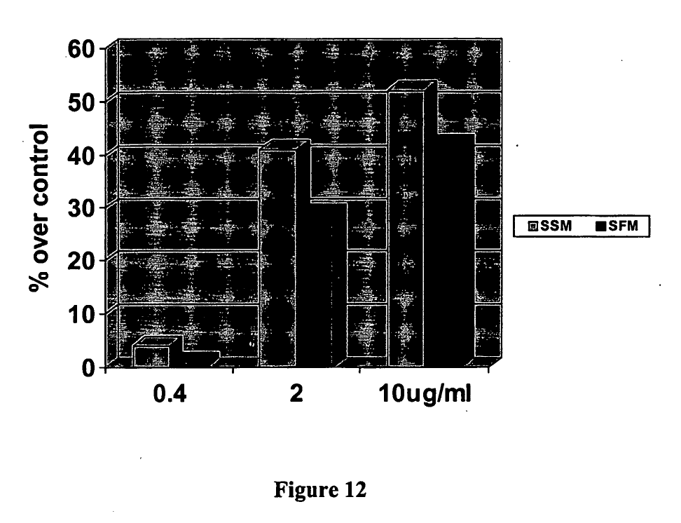

FIG. 12 shows the effect of permeate on glucose uptake in L6 cells. Cells were cultured, and treated in L6 Myo Medium (SSM)—SSM Protocol or in serum-free medium (SFM-DPBS containing 20 mM Hepes, 2 mM glucose)—SFM Protoci as described by Fryer et al. Glucose uptake was measured by adding 30 uM of 2-NBDG for 2′ at 22C. Experiments were performed in duplicates.

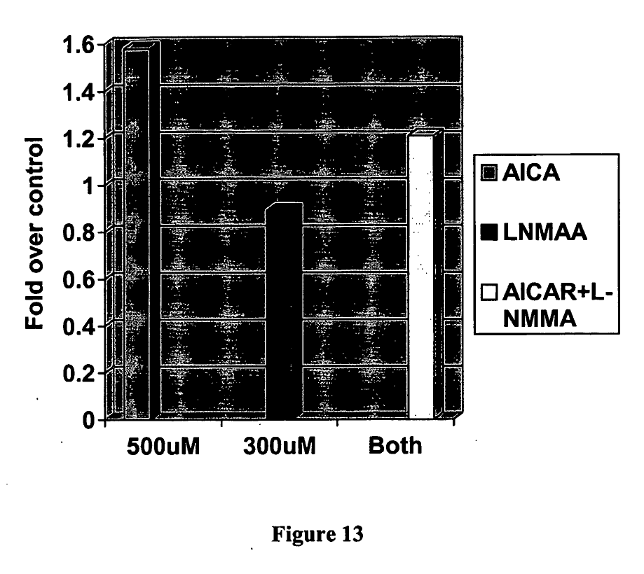

FIG. 13 shows the effect of AICA on the stimulation of glucose uptake and the effect of L-NMMA on the inhibition of glucose uptake. Similar effects are described by Fryer et al (Activation of glucose transport by AMP-activated protein kinase via stimulation of Nitric oxide synthase, Diabetes, 49(12), 1978-85, 2000, Fryer LGD, et al). The permeate cells were pre-treated with L-NMMA for 30′in DPBS and subsequently treated with AICA for 30 minutes. Glucose uptake was measured after the treatment in 22C for 2′.

In general, glucose transport in skeletal muscle is stimulated by two distinct stimuli, which are insulin and exercise. Recently it has been shown that AMP-activated protein kinase (AMPK) is activated by exercise in skeletal muscle. Pharmacological activation of AMPK by AICAR (5-amino-4-imidazolecarboxamide riboside) leads to an increase in glucose uptake. Interestingly, treatment of skeletal muscle cells with Nitric Oxide Synthase (NOS) inhibitors such as L-N-mono-methyl-L-arginine (1-NMMA) or L-N-nitro-L-arginine methyl ester (L-NAME) completely blocks the increase the glucose transport after activation of AMPK.

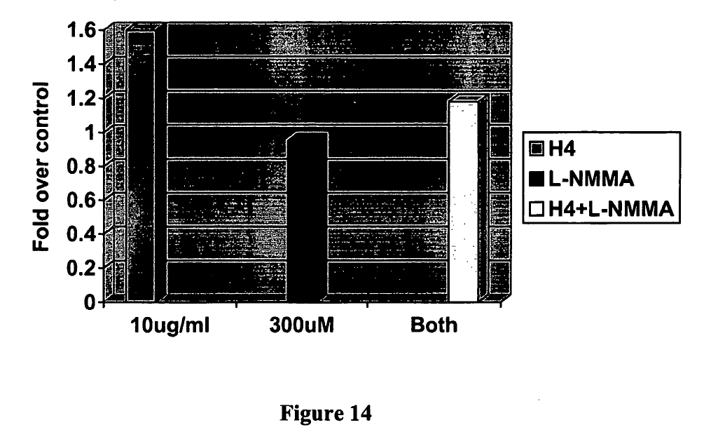

In the study represented by FIG. 14, AICA was used because AICAR is not commercially available. AICA is a precursor of AICAR and it is metabolized in muscle cells to AICAR resulting in increase glucose uptake.

L-NMMA has been used in the experiment to verify whether GMM-induced glucose transport in L6 cells is or is not inhibited by NOS inhibitors, thus mimicking AICAR—induced mechanism of increased glucose transport. Fryer's experimental set up was adopted, consisting of pre-treatment of cells with inhibitor for 30 minutes in DPBS and after that with agonists like AICAR or in our case GMM for next 30 minutes at 37C before performing glucose uptake.

In FIG. 14, permeate cells were tested to determine whether permeate-induced glucose uptake could be inhibited by L-NMMA at the same concentration as that used for inhibition of the AICA effect on glucose uptake. FIG. 14 shows that L-NMMA inhibits the effect of permeate on increased glucose uptake in L6 muscle cells in vitro. The L6 cells were pre-treated with L-NMMA in DPBS for 30 minutes and subsequently treated with GMM alone or GMM was added to cells treated with the inhibitor for next 30 minutes. Glucose uptake was measured at 22C for 2 minutes.

According to the results in FIG. 14, pretreatment of L6 cells with L-NMMA at a concentration of 300 uM blocks GMM-induced of glucose transport under these experimental conditions. It may suggest that GMM works through the AMPK pathway in a similar fashion to AICAR.

We claim:

1. A composition comprising a compound isolated from a plant, wherein the compound has AICAR (5′-aminoimidazole 4-carboxamide 1-ribonucleotide)-like activity and increases glucose uptake into a cell.

2. The composition of claim 1 wherein the plant is Hordeum vulgare and wherein the composition is isolated from a seed of the plant.

3. The composition of claim 1 having a molecular weight Mw of no more than 2000 and a UV light absorption maximum at about 260 nm.

4. The composition of claim 1 wherein the glucose uptake into the cell is increased when the composition is present in a medium surrounding the cell at a concentration of between about 2-10 micrograms per milliliter.

5. The composition of claim 4 wherein the cell or the medium surrounding the cell is depleted of at least one of a nutrient and oxygen.

6. The composition of claim 4 wherein the increase in glucose uptake is reduced when the medium comprises L-N-mono-methyl-L-arginine at a concentration of 300 micromole per liter.

7. The composition of claim 1 wherein the increase in glucose uptake into the cell is insulin independent.

8. The composition of claim 1 wherein the cell is a myocyte.

9. The composition of claim 1 wherein the cell is part of a tissue.

10. The composition of claim 9 wherein the tissue is in vivo and selected from the group consisting of liver tissue, skeletal muscle tissue, pancreatic tissue, and adipose tissue.

11. A composition comprising:
a compound having AICAR (5′-aminoimidazole 4-carboxamide 1-ribonucleotide)-like activity and wherein the compound increases glucose uptake into a cell;
wherein the compound is identical with a molecule isolated from Hordeum vulgare; and
wherein the molecule isolated from Hordeum vulgare reduces blood glucose in an organism when the molecule is administered to the organism at a concentration effective to reduce the concentration of glucose.

12. The composition of claim 11 wherein the molecule is isolated from a malted seed of Hordeum vulgare.

13. The composition of claim 11, wherein the isolation of the molecule from Hordeum vulgare comprises a procedure selected from the group consisting of malting, mashing, salt extraction, a buffer extraction, ethanol extraction, anion exchange chromatography, and molecular sieving.

14. The composition of claim 11 wherein the molecule has a molecular weight Mw of no more than 2000 and a UV light absorption maximum at about 260 nm, and wherein the glucose uptake into the cell is increased when the compound is present in a medium surrounding the cell at a concentration of between about 2-10 micrograms per milliliter.

15. The composition of claim 11 wherein the compound is synthesized in vitro.

16. The composition of claim 11 wherein the compound is modified to increase or decrease at least one of an increase in glucose uptake into a cell, solubility in a solvent, chemical stability, and in vivo specificity.

17. The composition of claim 11 further comprising a nutraceutical for treatment of diabetes.

18. The composition of claim 11 further comprising a pharmaceutical for treatment of diabetes.

19. A non-insulin compound that has AICAR (5′-aminoimidazole 4-carboxamide 1-ribonucleotide)-like activity and activates AMPK in a cell.

20. The compound of claim 19 wherein the compound is isolated from a plant seed.

21. The compound of claim 20 wherein the plant seed is from Hordeum vulgare.

22. The compound of claim 19 wherein the compound increases glucose uptake into a cell in a non-insulin dependent manner.

23. The compound of claim 22 wherein the glucose uptake into the cell is increased when the compound is present in a medium surrounding the cell at a concentration of between about 2-10 micrograms per milliliter.

24. The compound of claim 23 wherein the increase in glucose uptake is reduced when the medium comprises L-N-mono-methyl-L-arginine at a concentration of 300 micromole per liter.

25. The compound of claim 19 wherein the cell is selected from the group consisting of a hepatocyte, a myocyte, a pancreatic islet cell, and an adipocyte.

26. The compound of claim 25 wherein the cell is depleted of at least one of a nutrient and oxygen.

27. A method of treating a cell comprising:
identifying the cell as having a condition that activates an AMPK (adenosine 5′-monophosphate-activated protein kinase); and
presenting the cell with a compound having AICAR (5′-aminoimidazole 4-carboxamide 1-ribonucleotide)-like activity at a concentration effective to modulate at least one of an import, export, and synthesis of a molecule.
